(19) United States
(12) Patent Application Publication
(10) Pub. No.: US 2026/0130626 A1
(43) Pub. Date: May 14, 2026

(54) Joint Evaluation And Balancing

(71) Applicant: Howmedica Osteonics Corp., Mahwah, NJ (US)

(72) Inventors: Kevin Abbruzzese, Mahwah, NJ (US); Sally LiArno, Bergenfield, NJ (US); Justin Moidel, Verona, NJ (US)

(21) Appl. No.: 18/947,526

(22) Filed: Nov. 14, 2024

(51) Int. Cl. A61B 5/00; A61B 5/11; A61B 5/397; A61B 6/00; A61B 34/10; G16H 50/70

(52) U.S. Cl. CPC A61B 5/4851; A61B 5/1121; A61B 5/1128; A61B 5/397; A61B 5/6828; A61B 6/487; A61B 34/10; G16H 50/70; A61B 2034/105; A61B 2034/108

(57) ABSTRACT

Disclosed herein is a method and system for knee joint evaluation and balancing. The method can include the steps of tracking a knee joint during a patellar tendon reflex with a sensor to generate sensor data, generating image data related to the knee joint during the patellar tendon reflex, and determining a patellar tendon score from the sensor data and the imaging data. The patellar tendon score can represent a knee joint condition. The system can include a sensor configured to track a knee joint during a patellar tendon reflex, an imaging device configured to generate image data related to the knee joint, a processor in communication with the at least one sensor and the imaging device, and a user interface configured to display a patellar tendon score.

### FIELD OF THE DISCLOSURE

The present disclosure relates generally to methods and systems for joint evaluation and joint balancing, and in particular relates to methods and systems for evaluating a knee joint and knee joint balancing.

### BACKGROUND OF THE DISCLOSURE

Evaluating the condition of a knee joint for physical therapy or preparation for knee arthroplasty has traditionally relied on subjective assessments conducted manually by a health care provider (HCP). These assessments typically involve imaging techniques such as X-rays, MRI, or CT scans, as well as analog modalities like goniometers, to measure the knee joint's range of motion. However, these methods provide limited insight into the dynamic biomechanical properties of the knee due to their inherently subjective nature.

For instance, common clinical tests like the Lachman test, anterior/posterior drawer test, pivot shift test, quadriceps active test, and varus/valgus stress test are heavily dependent on the HCP's skill and experience. These tests aim to assess various aspects of knee function, including ligament integrity and joint stability, but they often fall short in quantifying critical biomechanical parameters such as stiffness, viscosity, damping, and other factors that influence knee joint health and performance.

Additionally, the accuracy of these tests can be significantly affected by external factors, including the patient's level of muscle relaxation and the HCP's subjective interpretation of resistance or movement during the test. This reliance on subjective interpretation leads to variability in assessments, which can result in inconsistent diagnoses and treatment plans.

Therefore, there is a need for improved methods and system that provides objective, quantifiable data on knee joint dynamics, leading to better-informed clinical decisions and improved patient outcomes.

### BRIEF SUMMARY OF THE DISCLOSURE

In certain embodiments, the present disclosure relates generally to a method for evaluating knee joint evaluation and balancing. Specifically, the disclosure broadly relates to systems and methods for evaluating a knee joint using objective, data-driven techniques. A system according to an embodiment of the present disclosure may integrate sensors and imaging devices to capture and process physiological, kinematic, and biomechanical data during specific joint reflexes or movements. The system may generate a quantitative assessment, such as a joint performance score, which represents the condition of the joint. The joint performance score may be utilized to guide clinical decisions across various phases of care, including pre-operative planning, intra-operative adjustments, and post-operative recovery. The system may further include components for data visualization, storage, and integration with databases for comparative analysis and long-term monitoring to facilitate personalized and optimized treatment strategies.

In accordance with an aspect of the present disclosure, a method to evaluate a knee joint is provided. A method according to this aspect, may include the steps of tracking a knee joint during a patellar tendon reflex using at least one sensor to generate sensor data, generating image data related to the knee joint during the patellar tendon reflex, and determining a patellar tendon score from the sensor data and the imaging data. The patellar tendon score may represent a knee joint condition.

Continuing in accordance with this aspect, the at least one sensor may include an electromyography (EMG) sensor configured to capture real-time muscle activation data during the patellar tendon reflex.

Continuing in accordance with this aspect, the at least one sensor may include an inertial measurement unit (IMU) configured to measure any of knee joint angles, velocity, and acceleration during the patellar tendon reflex.

Continuing in accordance with this aspect, the step of generating the image data may include capturing real-time kinematic data of the knee joint using computer vision.

Continuing in accordance with this aspect, the method may further include a step of calculating a knee joint stiffness parameter from the patellar tendon score.

Continuing in accordance with this aspect, the sensor data may include measurements from load cells to determine biomechanical forces acting on the knee joint during the patellar tendon reflex.

Continuing in accordance with this aspect, the method may further include a step of comparing the patellar tendon score with a pre-operative baseline data to evaluate recovery progress.

Continuing in accordance with this aspect, the method may further include a step of selecting an implant based on the patellar tendon score. The implant may be any of a femoral or tibial implant for the knee joint. The method may further include a step of positioning the implant based on patellar tendon score.

Continuing in accordance with this aspect, the image data may include fluoroscopic images of patellar alignment during the patellar tendon reflex.

Continuing in accordance with this aspect, the method may further include the step of integrating the patellar tendon score with a database of patellar tendon scores for comparative analysis across patient populations.

Continuing in accordance with this aspect, the method may be performed pre-operatively, intra-operatively, and post-operatively to provide continuous assessment of the knee joint.

Continuing in accordance with this aspect, the method may further include a step of outputting the patellar tendon score on a user interface. The user interface may display any of a reflex speed, peak extension, and range of motion of the knee joint.

Continuing in accordance with this aspect, the at least one sensor may include a wearable device configured to provide real-time feedback during the patellar tendon reflex.

In accordance with another aspect of the present disclosure, a system for evaluating a knee joint is provided. A system according to this aspect, may include at least one sensor configured to track a knee joint during a patellar tendon reflex and generate sensor data, an imaging device configured to generate image data related to the knee joint during the patellar tendon reflex, a processor in communication with the at least one sensor an the imaging device, the processor may be configured to receive the sensor and the image data and determine a patellar tendon score based on the sensor data and the image data, and a user interface configured to display the patellar tendon score. The patellar data score may represent a knee joint condition.

Continuing in accordance with this aspect, the at least one sensor may include an electromyography (EMG) sensor configured to capture muscle activation data during the patellar tendon reflex.

Continuing in accordance with this aspect, the processor may further be configured to calculate a knee joint stiffness parameter from the patellar tendon score.

Continuing in accordance with this aspect, the at least one sensor may include an inertial measurement unit (IMU) configured to measure any of a knee joint angle, velocity, and acceleration during the patellar tendon reflex.

Continuing in accordance with this aspect, the processor may be configured to integrate the patellar tendon score with a pre-operative baseline data to evaluate changes in knee joint performance over time.

Continuing in accordance with this aspect, the user interface may be configured to display additional metrics derived from the patellar tendon reflex including any of a reflex speed, peak extension, and range of motion of the knee joint.

Continuing in accordance with this aspect, the processor may be further configured to store the patellar tendon score and the additional metrics in a database for comparative analysis across patient populations.

In accordance with another aspect of the present disclosure, a method for balancing a knee joint is provided. A method according to this aspect, may include the steps of obtaining imaging data of a knee joint, generating a model of the knee joint, conducting a motion analysis of the knee joint by tracking relative motion between a tibia and a femur to generate a motion arc data, determining a center of rotation (COR) axis of the tibia relative to the femur based on the motion arc data, positioning a femoral trial to align a central axis of the femoral trial with the COR axis, positioning a tibial trial in a first position, assessing ligament tension of the knee joint, and selecting a femoral implant based on a natural joint line of the knee joint.

Continuing in accordance with this aspect, the method may further include a step of adjusting the tibial trial in six degrees of freedom such that the ligament tension is balanced.

Continuing in accordance with this aspect, the imaging data may include CT scans segmented to create a three-dimensional model of the knee joint.

Continuing in accordance with this aspect, the step of conducting a motion analysis may include tracking points on the tibia relative to the femur during a pendulum knee drop.

Continuing in accordance with this aspect, the step of determining the COR axis may further include generating best-fit circles from the motion arc data, and calculating the COR axis as a line passing through center points of the best-fit circles.

Continuing in accordance with this aspect, the method may further include the step of virtually positioning the femoral trial and tibial trial in a simulated environment prior to performing an intraoperative assessment.

Continuing in accordance with this aspect, the step of assessing ligament tension may include using a robotic tensioning device to measure soft tissue tension at multiple points across a flexion arc.

Continuing in accordance with this aspect, the step of selecting the femoral implant may include selecting a femoral implant with a single flexion radius to maintain ligament isometry across the flexion arc.

Continuing in accordance with this aspect, the step of adjusting the tibial trial in six degrees of freedom may include modifications in any of a varus/valgus alignment, anterior/posterior translation, and rotational positioning.

Continuing in accordance with this aspect, the method may include a step of selecting medial and lateral tibial inserts with differing thicknesses to achieve balanced ligament tension is ligament asymmetry exists.

Continuing in accordance with this aspect, the method may include a step of preoperatively analyzing motion data using fluoroscopy or a vision-based system to determine the COR axis prior to trialing.

Continuing in accordance with this aspect, the step of generating the motion arc data may include tracking anatomical landmarks. The anatomical landmarks may include medial and lateral resection points and a tibial knee center.

Continuing in accordance with this aspect, may include a step of aligning the tibial trial to the COR axis prior to assessing ligament tension.

Continuing in accordance with this aspect, the ligament tension may be assessed in both medial and lateral compartments to ensure balanced tension throughout a range of motion of the knee.

### BRIEF DESCRIPTION OF THE DRAWINGS

A more complete appreciation of the subject matter of the present disclosure and the various advantages thereof may be realized by reference to the following detailed description, in which reference is made to the following accompanying drawings:

FIG. 1 is a schematic view of a system for generating a patellar tendon score according to an embodiment of the present disclosure;

FIG. 2 is a graph showing a patellar tendon reflex according to an embodiment of the present disclosure;

FIG. 3 is a graph showing an EMG curve during a patellar tendon reflex according to an embodiment of the present disclosure;

FIG. 4 is a first view of user interface of the system of FIG. 1 according to an embodiment of the present disclosure;

FIG. 5 is a second view of the user interface of FIG. 4;

FIG. 6 is a system for generating a knee joint score according to an embodiment of the present disclosure;

FIG. 7 is a flowchart showing steps for a knee joint balancing method according to an embodiment of the present disclosure;

FIG. 8 is schematic diagram of a knee joint motion for determining a center of rotation according to an embodiment of the present disclosure;

FIG. 9 is a sagittal view of a knee joint showing the center of rotation according to an embodiment of the present disclosure, and

FIG. 10 is bottom view of a distal femur of the knee joint of FIG. 9 showing the center of rotation.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described. As used herein, the terms “knee” and “joint” will be used interchangeably and as such, unless otherwise stated, the explicit use of either term is inclusive of the other term.

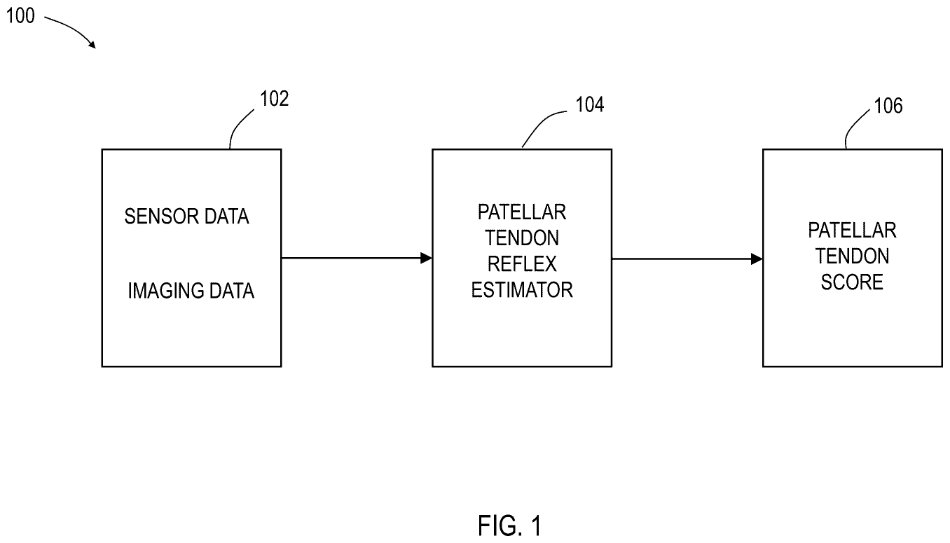

FIG. 1 shows a system 100 for generating a patellar tendon score 106 according to an embodiment of the present disclosure. A patellar tendon reflex estimator 104 is used to generate patellar tendon score 106 for assessing neurological and biomechanical aspects of knee joint health including quadriceps efficiency and the performance of the extensor mechanism. Patellar tendon score 106 provides an objective measure of the knee's response to patellar tendon reflex (knee-jerk reflex) and helps evaluate critical indicators such as muscle strength, movement stability, and dynamic joint performance for informed clinical decisions in procedures like total knee arthroplasty (TKA) and rehabilitation. Patellar tendon score 106 offers HCPs a data-driven approach to assess knee joint functionality, moving beyond traditional subjective measures and providing a more objective, repeatable method for evaluating key aspects of knee dynamics and performance using patellar tendon reflex.

Input data 102 for system 100 can include a diverse array of data types derived from one or more sensors and imaging modalities during patellar tendon reflex. Each input contributes insights into the joint's physical and neurological functions. Sensor data collected from multiple types of devices provides a detailed picture of knee mechanics, muscle activation, and movement patterns during patellar tendon reflex. For example, electromyography (EMG) sensors are employed to capture real-time data on muscle activation within the quadriceps, delivering information about muscle contraction patterns, activation timing, and overall strength. By evaluating these characteristics, system 100 assesses quadriceps efficiency, detecting potential muscle weaknesses or imbalances that could impact joint stability and function. This information can be used in post-operative assessments and ongoing rehabilitation, as it offers insights into muscle recovery and the effectiveness of physical therapy regimens.

Input data 102 for system 100 can include information from Inertial Measurement Units (IMUs), which can be integrated into wearable devices, implant, or trials, to record motion data, such as joint angles, velocity, and acceleration during knee movements. IMUs can provide kinematic data that helps track range of motion, assess movement quality, and detect irregularities during patellar tendon reflex, which could indicate issues with joint stability, coordination, or potential compensatory movements. The motion data gathered from IMUs is beneficial when combined with the patellar tendon reflex arc, as it allows the system to analyze specific kinematic responses, such as oscillations and decay rates offering insight into how efficiently the extensor mechanism is functioning, which can be helpful for evaluating recovery progress and guiding decisions on surgical intervention or targeted therapy.

In addition to IMUs and EMG sensors, computer vision systems integrated into system 100 can enable real-time kinematic analysis of limb movement and anthropometric measurements, capturing factors such as limb alignment, dynamic positioning, and anatomical characteristics that influence knee function. Through advanced imaging and AI processing, computer vision systems can track the movement of the patella and surrounding structures during patellar tendon reflex, allowing system 100 to evaluate how these factors impact joint mechanics and overall knee performance. Load cells can be used to measure forces and torque at various points, providing a biomechanical profile of load distribution across the knee joint during patellar tendon reflex. This measurement helps system 100 to identify the mechanical stresses placed on the knee joint for tailoring rehabilitation programs and for making informed adjustments to surgical techniques, if necessary.

System 100 can also incorporate imaging data to utilize structural and functional view of the knee as input data 102 in other embodiments. For instance, fluoroscopy can be used to capture real-time visualization of patellar movement to input assessment of joint alignment and tracking during patellar tendon reflex. Additionally, CT scans from bone databases can be used as input data 102 for detailed structural characteristics of the patella and surrounding bones. DEXA scans can offer a layer of information on bone mineral density, which can be used by system 100 for evaluating bone health and determining the risk of fracture or implant stability, especially in patients undergoing or recovering from knee replacement surgery.

By combining data from one or more of EMG muscle activity, IMU-recorded kinematic performance, computer vision-based kinematics, and various imaging modalities, system 100 can deliver an objective assessment of knee joint condition during patellar tendon reflex including extensor mechanism performance. This data-rich approach is configured to support clinical decision-making throughout the entire episode of care, from initial assessments and surgical planning to rehabilitation and ongoing patient monitoring. The combined insights from muscle activity and joint motion during patellar tendon reflex can inform treatment pathways, helping guide choices around rehabilitation methods, surgical techniques, and specific patient needs, such as implant selection, quadriceps management (e.g., incision planning), and patella positioning (e.g., alta vs. baja).

System 100 can integrate one or more of the multiple data sources, including sensors, kinematic measurements, and imaging modalities to evaluate the performance of the extensor mechanism and patella-femoral kinematics. Thus, system 100 provides quantifiable data to allow for precise evaluations of anatomical efficiency, neuromuscular activation, and patient-specific knee performance, contributing to more tailored and effective clinical interventions.

Patellar tendon score 106 can serve as a tool for knee joint evaluation across distinct phases of surgical intervention including pre-operative (pre-op), intra-operative (intra-op), and post-operative (post-op) phases. By systematically applying patellar tendon score 106 at each phase, system 100 provides a continuous, objective method for assessing and guiding patient care, facilitating data-driven clinical decisions throughout the entire treatment cycle.

In the pre-op phase, patellar tendon score 106 can be used to establish a detailed baseline of the knee joint's functional status. Specifically, the score provides insights into quadriceps efficiency, neuromuscular response characteristics, and the biomechanical integrity of the extensor mechanism. These measurements can be derived from quantitative data on muscle activation, joint kinematics, and load distribution as input data 102, offering a clear profile of the patient's knee joint performance. This baseline data can be used in surgical planning, enabling the HCP to identify specific functional or structural conditions that may influence the approach to surgery. For instance, the pre-operative patellar tendon score 106 can help determine if adjustments to alignment, implant choice, or muscle management strategies are warranted to achieve the best possible outcomes.

During the intra-op phase, patellar tendon score 106 can provide real-time assessments of patella-femoral tracking and extensor mechanism performance. Dynamic measurements, facilitated by robotic systems in some embodiments, can enable HCPs to evaluate kinematics and understand how surgical decisions impact implant performance and joint mechanics. Patellar tendon score 106 offers precise data on patella alignment, soft tissue balance, and implant placement, enabling the HCP to adjust intra-operatively for optimal outcomes. For example, fluoroscopy or similar imaging modalities can complement the patellar tendon score 106 assessments by providing detailed visualizations of patella kinematics during surgery, enhancing the precision of alignment and balance adjustments.

In the post-op phase, patellar tendon score 106 can support ongoing monitoring of the extensor mechanism and patella-femoral performance, providing objective data for tracking recovery. Patellar tendon score 106 indicates changes in extensor mechanism performance over time, allowing HCPs to evaluate deficiencies, instability, or implant mechanics. These measurements can be compared with patient-reported outcome measures (PROMs) to provide a more comprehensive view of recovery progress. Additionally, repeated assessments using patellar tendon score 106 help monitor patient performance, document progress, and refine rehabilitation plans to address individual needs. The ability to continuously assess the extensor mechanism ensures that potential issues, such as instability or delayed recovery, are identified and addressed promptly.

Patellar tendon score 106 collected from a patient or multiple patients can be used to create a database to catalog reflex values and extensor mechanism responses across all phases of care. This database can serve as a tool for characterizing patients and refining clinical decisions based on clusters of extensor mechanism performance, implant type, and PROMs. By establishing a closed-loop feedback system, patellar tendon score 106 facilitates a data-driven approach to improving TKA planning, rehabilitation strategies, and patient outcomes. This iterative process allows clinicians to identify trends and optimize interventions, tailoring care to meet the specific needs of individual patients.

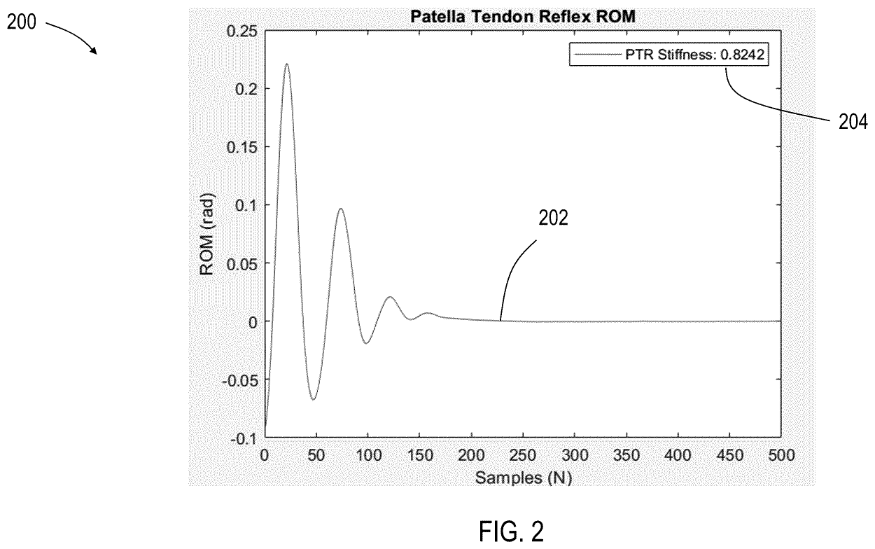

FIG. 2 shows a graph 200 representing a patellar tendon reflex range of motion (ROM) over time, illustrating the oscillatory response of the knee joint following a reflex stimulation event. The x-axis indicates the samples (N), representing time progression, while the y-axis represents the range of motion (rad) in radians in graph 200. Curve 202 corresponds to the dynamic movement response of the knee joint during the patellar tendon reflex.

As shown in graph 200, ROM registers a significant initial oscillation, indicating the reflex-induced movement of the knee joint caused by the stimulus. This peak reflects the immediate angular displacement of the knee joint in response to the reflex. As time progresses, the oscillations gradually decay, stabilizing to a near-zero range of motion, signifying the return of the knee joint to its equilibrium position.

A calculated patellar tendon stiffness 204 value is shown in graph 200, which quantifies the resistance and stability of the extensor mechanism during and after the reflex event. Patellar tendon stiffness 204 provides an objective measure of the biomechanical properties of the knee joint, such as damping and stiffness, which are indicative of joint health and extensor mechanism performance.

The decaying oscillations shown in FIG. 2 provide key data on the knee joint's dynamic response. This data allows system 100 to assess parameters such as joint stability, neuromuscular efficiency, and the functional characteristics of the extensor mechanism and output a patellar tendon score 106. Curve 202 also serves as a basis for deriving additional insights, including the reflex decay rate and oscillation frequency, which can be used to monitor recovery progress, evaluate implant effectiveness, and guide rehabilitation strategies.

System 100 can utilize EMG measurements as input data 102 to assess muscle activity and neuromuscular performance to generate patellar tendon score 106 in one embodiment. The EMG measurement process can include the application of surface electrodes to the skin over specific muscles to detect the electrical activity generated during muscle contraction during patellar tendon reflex. These surface electrodes measure the voltage difference between applied electrical leads, providing real-time data on motor unit recruitment and muscle activation patterns.

EMG can serve as a screening tool to evaluate the quadriceps' response prior to surgery. This pre-operative assessment can inform surgical decisions, such as the method of incision or the management of surrounding soft tissues. Additionally, EMG can be used to evaluate antagonist muscles, such as the hamstrings, to understand complementary muscle dynamics and overall joint stability during patellar tendon reflex. By assessing both primary and antagonist muscles, the system captures a comprehensive view of the neuromuscular contributions to knee joint performance.

System 100 can record EMG signals during both active and passive activities. For example, during passive conditions, EMG measurements provide insights into muscle contributions to stability, even in the absence of voluntary contraction. This can be particularly useful for assessing baseline neuromuscular function or for identifying potential deficiencies in muscle engagement.

The amplitude of the EMG signal represents the degree of motor unit recruitment. Higher amplitudes correspond to greater muscle activation and strength, especially during activities such as maximum voluntary muscle contraction (MVMC). These measurements can be used as a surrogate for overall muscle strength, helping to characterize the performance of specific muscles, such as the quadriceps, during different phases of care.

EMG data can be collected pre-operatively to establish a baseline for muscle function and post-operatively to monitor recovery and improvements in muscle performance. This analysis can allow HCPs to track changes in neuromuscular activation, identify trends in muscle recovery, and adjust rehabilitation protocols as needed. By combining EMG measurements with other input data 102, system 100 provides a framework for evaluating the neuromuscular and biomechanical aspects of knee joint health, ensuring personalized care throughout the patient's treatment and recovery process.

EMG measurements can be integrated with system 100 to enhance knee joint evaluation and management across the pre-op, intra-op, and post-op phases. In the pre-op phase, EMG measurements capture the electrical response during patellar tendon reflex to estimate muscle strength and neuromuscular function. This data provides HCPs with quantitative insights into the extensor mechanism's baseline performance, enabling patient characterization and planning. EMG data can be incorporated into predictive algorithms to recommend prehabilitation strategies or optimize surgical techniques, such as selecting between muscle-sparing or sacrificing incision methods (e.g., medial parapatellar, midvastus, or subvastus approaches). Additionally, EMG measurements help quantify reflex performance and characterize the patella-femoral response, guiding implant sizing and positioning decisions for improved extensor mechanism performance.

During surgery, EMG can serve as a proxy for assessing muscle strength and function, directly informing critical decisions regarding the surgical approach and implant placement. For example, real-time EMG data can guide the selection of incision techniques, ensuring that muscle integrity is preserved or balanced to optimize recovery potential. Similarly, EMG measurements can assist in determining the optimal implant size and alignment to enhance the biomechanical performance of the extensor mechanism. This real-time feedback allows for tailored surgical interventions that address individual patient needs.

In the post-op phase, EMG measurements can be used to track recovery progress by monitoring changes in muscle activation and strength over time. By comparing pre-operative and post-operative EMG data, HCPs can evaluate improvements in neuromuscular function and adjust rehabilitation protocols to support optimal recovery outcomes. EMG data can be integrated with patellar tendon score 106 to develop predictive algorithms that complement surgical decisions and monitor long-term recovery trends. For instance, repeated EMG assessments can identify deficiencies in muscle performance or stability, prompting timely interventions to ensure recovery milestones are met.

EMG measurements, combined with patellar tendon score 106 data, can be stored in a centralized database, allowing HCPs to catalog reflex values and extensor mechanism responses across patient populations. This database supports the development of predictive models and clinical decision-making tools that enhance the continuum of care. Clustering patients based on reflex responses, neuromuscular performance, and surgical outcomes enables refinement of treatment protocols, improving both short-term and long-term patient outcomes.

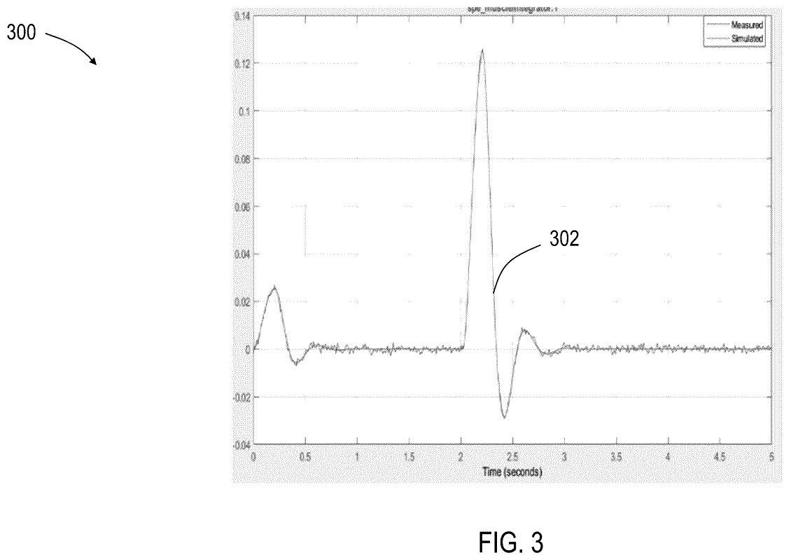

Referring now to FIG. 3, there is shown a graph 300 depicting a simulated EMG response of the quadriceps muscle during patella tendon reflex. The x-axis represents time (seconds), capturing the temporal progression of the reflex response, while the y-axis shows the amplitude (voltage), indicating the electrical activity recorded during patellar tendon reflex. Plotted curve 302 represents the EMG data, with two distinct curves. A first curve represents the measured response, and the second curve represents the simulated response.

At approximately 2.5 seconds, a pronounced peak in amplitude is observed in graph 300 corresponding to the reflex-induced contraction of the quadriceps muscle. This peak reflects the maximum motor unit recruitment and electrical activity generated during the patella tendon reflex. The sharp increase in amplitude indicates a strong neuromuscular response, which is followed by a gradual decay as the reflex subsides and the muscle returns to a baseline state. The baseline activity, observed before and after the reflex event, reflects the steady-state electrical activity of the muscle under passive conditions.

Graph 300 provides insights into the reflex behavior and neuromuscular function of the extensor mechanism according to an embodiment of the present disclosure. The peak amplitude during the reflex represents the strength and efficiency of the quadriceps contraction, which can be used to assess muscle performance and neurological response. By comparing the measured response with the simulated response, HCPs can validate the accuracy of the EMG data and the reliability of predictive models.

The EMG response shown in FIG. 3 can be integrated with other input data 102, such as kinematic data or reflex scores to provide a complete assessment of the extensor mechanism. For example, the timing and amplitude of the reflex response can be used to characterize neuromuscular function pre-operatively, guide intra-operative adjustments, or track post-operative recovery. Additionally, the observed patterns in reflex decay and baseline recovery can inform assessments of joint stability, muscle strength, and rehabilitation progress.

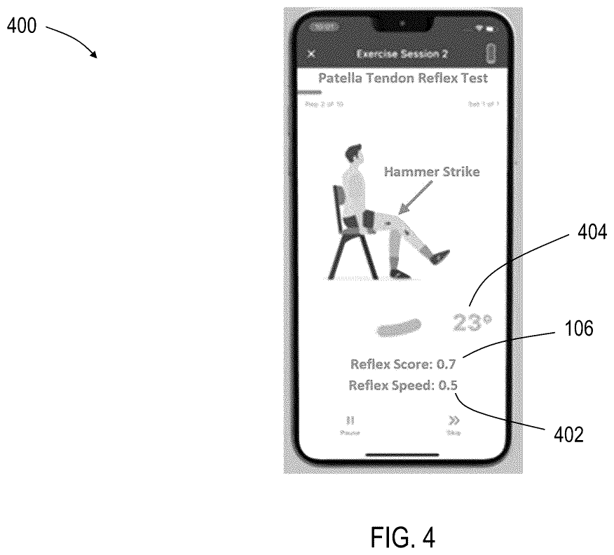

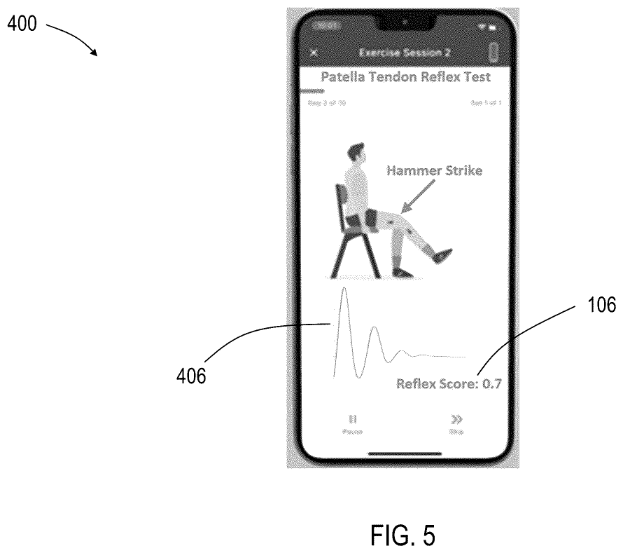

FIGS. 4 and 5 show a user interface 400 for system 100 with outputs from patellar tendon reflex estimator 104 according to an embodiment of the present disclosure. Interface 400 can be displayed on a smartphone, tablet, computer, smartwatch, etc. Interface 400 is configured to visually represent patellar tendon reflex parameters in real-time, offering an intuitive platform for HCPs and patients to monitor reflex-related metrics. The display can include key data points such as patellar tendon score 106, reflex speed 402, peak extension 404, and reflex kinematics 406 like ROM, all derived from patellar tendon reflex.

FIG. 4 shows a hammer strike triggering the patellar tendon reflex, with corresponding feedback on reflex performance. The displayed patellar tendon score 106 quantifies the overall neuromuscular response, while reflex speed 402 provides insights into the latency and velocity of the muscle's response to the stimulus. Additionally, interface 400 visually illustrates the degree of peak extension 404, offering a clear measure of the knee joint's immediate range of motion resulting from the reflex.

FIG. 5 shows a graphical representation of reflex kinematics 406, specifically the oscillatory motion captured during the test. This waveform visualization complements patellar tendon score 106, illustrating the dynamic behavior of the knee joint over time, including the amplitude and decay of the reflex response. Together, these features enable a complete evaluation of reflex activity, combining quantitative scores with graphical kinematic data for enhanced understanding.

Interface 400 is tailored for usability, providing HCPs and patients with actionable data to assess neuromuscular function and joint performance. By integrating reflex information with kinematic measures, system 100 supports both immediate assessments, such as during clinical exams, and longitudinal monitoring across pre-operative, intra-operative, and post-operative phases of care.

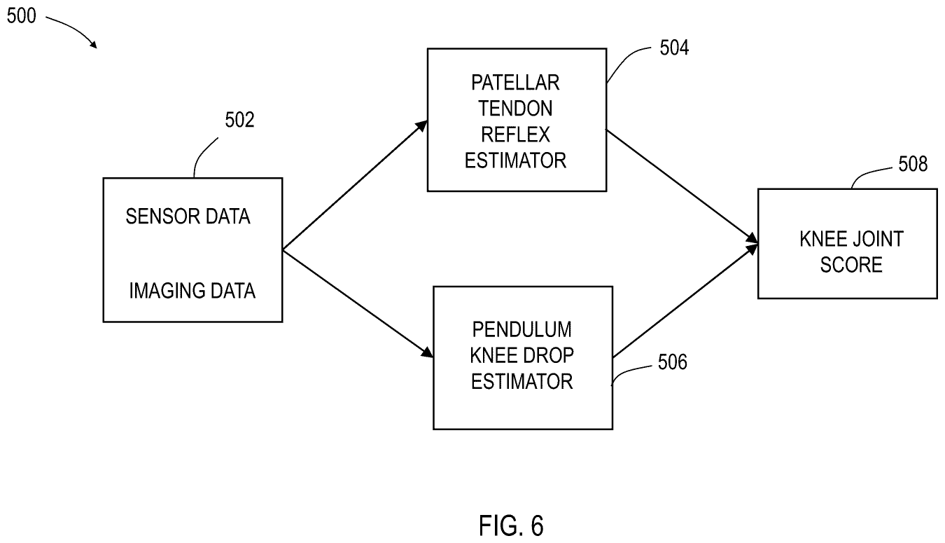

FIG. 6 shows a system 500 for conducting passive knee kinematic evaluation according to an embodiment of the present disclosure. The passive knee kinematic evaluation can be conducted throughout the episode of care for TKA to provide quantifiable and reproducible data across pre-op, intra-op, and post-op phases. System 500 integrates objective assessment techniques, including the Pendulum Knee Drop (PKD) test and the patellar tendon reflex. System 500 can utilize robotic systems, IMUs, and computer vision, to address the limitations of traditional subjective methods for evaluating knee laxity, stiffness, and performance.

As shown in FIG. 6, system 500 can evaluate knee joint performance by integrating sensor data and imaging data, processed through two computational components: a patellar tendon reflex estimator 504 and a pendulum knee drop estimator 506. The combined outputs from these components are used to generate a knee joint score 508, quantifying the biomechanical, neuromuscular, and functional characteristics of the knee joint across the episode of care.

System 500 can receive one or more inputs 502 from sensor data and imaging data. Sensor data can include inputs from EMG sensors, IMUs, load cells, and computer vision markerless motion capture technologies, etc. These modalities capture dynamic parameters such as ROM, oscillation patterns, joint forces, stiffness, and muscle activation. Imaging data, such as CT scans, fluoroscopy, and DEXA scans, provide structural and anatomical insights into the knee joint, including 3D models for analyzing bone structure, cartilage integrity, and soft tissue alignment. Together, these inputs offer a complete view of the knee's structural and functional attributes.

Patellar tendon reflex estimator 504 processes sensor and imaging data to evaluate neuromuscular responses and extensor mechanism performance. This component calculates metrics such as reflex speed, oscillation decay, muscle activation, joint stiffness, and torque. These reflex-derived assessments provide insights into the interplay between neurological and mechanical responses of the knee joint. Complementing this, pendulum knee drop estimator 506 analyzes passive knee motion data captured during the pendulum knee drop test. This estimator evaluates intrinsic properties such as joint stiffness, viscosity, passive ROM, and oscillatory behavior using data from IMUs, computer vision systems, and robotic optical navigation technologies. Together, the estimators provide a complete analysis of both active and passive biomechanical performance to compute knee joint score 508. By integrating multi-modal data through a closed-loop process, system 500 provides an objective framework for knee joint evaluation, enhancing clinical decision-making and enabling personalized care throughout the TKA episode of care.

In the pre-op phase, system 500 can combine CT-derived insights with passive kinematic evaluations such as the PKD and patellar tendon reflex tests to establish a detailed baseline of knee function. A CT scan can be performed to create a 3D virtual model of the joint, which allows clinicians to evaluate bone structure, disease severity, and joint alignment while estimating soft tissue parameters such as cartilage condition and osteophyte formation.

The PKD test, a gravitational assessment technique, measures knee stiffness and viscosity by positioning the leg near full extension and allowing it to swing passively. Data from IMUs or computer vision systems capture the oscillations, range of motion (ROM), and decay rates, providing quantitative measures of joint laxity and soft tissue behavior. The patellar tendon reflex complements these measurements by assessing neurological responses related to quadriceps efficiency and extensor mechanism performance. EMG data from the reflex test can be used to measure muscle strength, offering additional insights into the extensor mechanism's condition. This combination of kinematic data, reflex measurements, and CT-derived parameters supports pre-operative decisions, such as implant sizing, quadriceps incision planning, and estimation of knee stiffness. These insights can be used as inputs for robotic-assisted TKA planning, enabling data-driven surgical strategies.

During the intra-op phase, system 500 can integrate pre-op data with real-time measurements to guide surgical decisions. Using robotic-assisted TKA devices and optical navigation systems, HCPs can perform additional PKD assessments to confirm pre-operative kinematic findings prior to incision. Anatomical landmarks registered via robotics can be paired with CT-derived native anatomy, providing a detailed understanding of knee performance differences between the diseased and functional states.

Intra-op PKD assessments can be used to refine surgical decisions, such as the selection of incision techniques (e.g., medial parapatellar, midvastus, subvastus), implant trialing, and soft tissue balancing. These evaluations provide additional data on knee stiffness, laxity, and soft tissue tension, informing adjustments like insert thickness, tibial slope modification, and soft tissue releases. The PKD test, in conjunction with robotic optical navigation, offers real-time feedback on passive knee kinematics, enabling HCPs to optimize implant positioning and alignment for stability and functional restoration.

System 500 can also include additional laxity tests, such as AP drawer and varus/valgus assessments, to provide a comprehensive evaluation of ligament tension and overall knee stability. The intra-op data can be cataloged in a database, creating a closed feedback loop that enhances robotic planning, implant selection, and physical therapy recommendations for future patients.

Post-operatively, system 500 can continue to monitor changes in knee stiffness and performance using the PKD and patellar tendon reflex. These objective measurements can be compared to PROMs and used to evaluate recovery progress. System 500 can characterize inflammation, instability, and implant mechanics over time, documenting changes in joint performance and identifying potential issues requiring intervention. The post-op phase can leverage the same kinematic and reflex metrics to adjust rehabilitation protocols, ensuring individualized care.

The combination of kinematic parameters (e.g., ROM, stiffness, torque), reflex data, and robotic insights allows for the creation of a closed-loop process that spans all phases of care. System 500 not only enables precise assessments of knee joint function but also informs decisions related to implant selection, surgical techniques, and rehabilitation strategies.

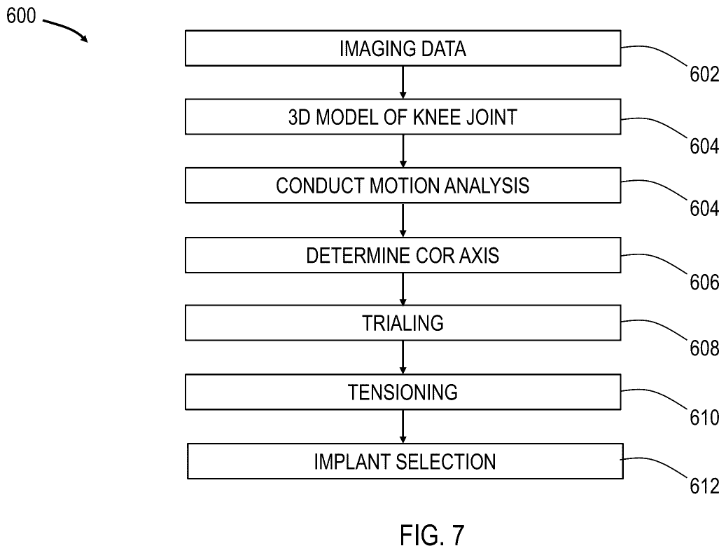

FIG. 7 is a flowchart 600 showing steps for a knee joint balancing method according to an embodiment of the present disclosure. Knee joint instability remains a leading cause of aseptic revision in TKA. Achieving proper knee balancing to prevent instability is a critical goal during surgical planning and execution. Conventional techniques focus on ligament tension and implant positioning but fail to account for the center of rotation (COR) of the tibia relative to the femur. This limitation can result in suboptimal joint stability, reduced implant longevity, and lower patient satisfaction. Flowchart 600 directly addresses this shortcoming by accurately determining and aligning the COR with a central axis of the femoral implant. Flowchart 600 discloses a method for balancing the knee joint in TKA by determining and aligning the COR of the tibia relative to the femur. This method can involve intraoperative or preoperative steps to calculate the COR axis for precise placement of the femoral implant. The method utilizes a femoral implant with a single flexion radius through the arc of motion where soft tissues are isometric. By aligning the central axis of the femoral implant to the COR axis, tibial adjustments alone suffice to achieve knee balance. This approach ensures ligament isometry, implant stability, and optimized biomechanics throughout the range of motion.

In a step 602, imaging data of the patient's knee joint anatomy is obtained. This can be obtained through any means such as a computed tomography (CT) scan. The image data can include detailed, three-dimensional representation of the bony structures of the femur and tibia. Alternative imaging modalities, such as MRI, may also be used depending on the clinical requirements.

Using the imaging data from step 602, a three-dimensional (3D) model of the patient's knee joint is created in a step 604. The 3D model represents the spatial relationships of key anatomical landmarks, including the femoral condyles and tibial plateau. The 3D model enables virtual planning by providing a detailed visualization of the knee joint geometry for accurate alignment and surgical decision-making.

Motion analysis of the knee joint can then be conducted in a step 606 to understand the relative movement between the tibia and femur. This step can be performed intraoperatively using robotic systems or optical tracking systems. The knee is put through controlled motions, such as a pendulum knee drop test or a guided range of motion. During these movements, trackers applied to the femur and tibia can capture real-time kinematic data. This data includes the arc of motion, ligament behavior, and joint dynamics, forming the basis for determining the COR. If preoperative analysis is performed, active patient motions (e.g., walking, squatting, stair ascent/descent) can be recorded using vision-based systems or fluoroscopy.

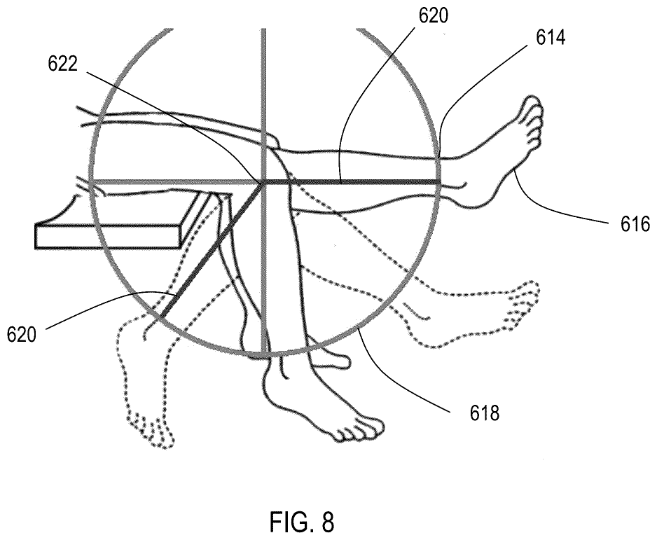

In a step 608, the COR axis of the tibia relative to the femur is determined from the motion analysis conducted in step 606. Step 608 includes tracking specific anatomical landmarks, such as the medial and lateral resection points and the tibial knee center. By mapping the motion arcs (e.g., lateral malleoli movements) and creating best-fit circles for the paths of these landmarks, the COR axis is identified. This axis represents the rotational center around which the tibia moves relative to the femur, forming the critical reference for implant alignment. FIG. 8 shows a schematic representation of the process used to calculate the COR of the tibia relative to the femur during knee joint motion. Key anatomical landmarks are tracked and analyzed during this motion to determine the COR.

FIG. 8 shows a schematic of the lower limb, including a femur, a tibia, and a foot 616, undergoing a controlled motion, such as a pendulum knee drop. The knee is represented at various positions along its range of motion as indicated by the dashed outlines of the leg. The top of a lateral malleolus 614, located near the ankle, is used as a tracking point on the tibia. The relative motion of this point with respect to the femur is analyzed throughout the arc of motion. The boundaries of the motion arc are indicated by lines 620.

A circle 618 represents the best-fit circle created from the motion arc. A center of this circle corresponds to a COR 622, which is the rotational center of the tibia relative to the femur during the observed motion. COR 622 serves as the reference for aligning the central axis of the femoral implant. This alignment ensures that the implant is positioned to replicate the natural rotational mechanics of the knee joint.

Virtual trialing can then be performed to simulate the placement of the femoral implant in a step 610. The virtual femoral trial is positioned such that the axis passing through the center of its medial and lateral condyles aligns with the COR axis determined in step 608. A tibial trial is then placed in a default starting location. This trialing step provides an initial evaluation of implant positioning and alignment, ensuring that the COR is preserved.

Ligament tensioning is then performed to assess soft tissue tension in the medial and lateral compartments of the knee in a step 612. The goal is to evaluate and achieve equal tension across the flexion arc, ensuring ligament isometry. Step 612 can involve robotic tensioners or manual methods to measure ligament forces at multiple points of the arc of motion. The tensioning process provides quantitative data to guide further adjustments, particularly to the tibial component's positioning.

Based on the trialing and tensioning results, the appropriate femoral implant is selected in a step 612. This implant is chosen to most closely recreate the patient's natural joint line while maintaining alignment with the COR axis. Adjustments to the tibial implant are made in six degrees of freedom (e.g., including varus/valgus, anterior/posterior, proximal/distal, and rotational alignment) to achieve a fully balanced knee. If necessary, separate medial and lateral inserts can be used to address asymmetrical soft tissue tension.

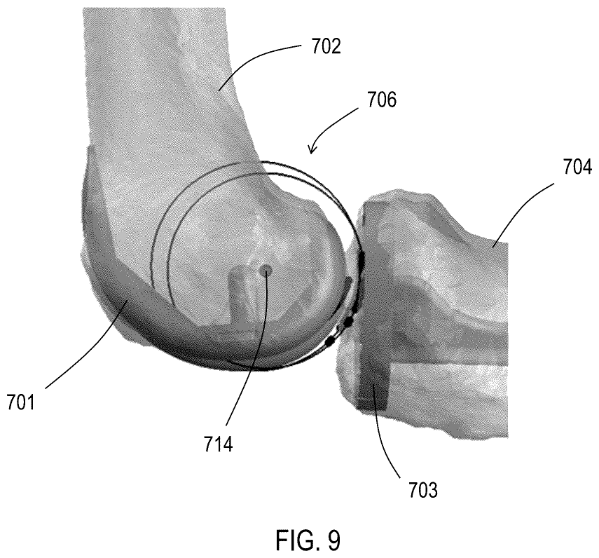

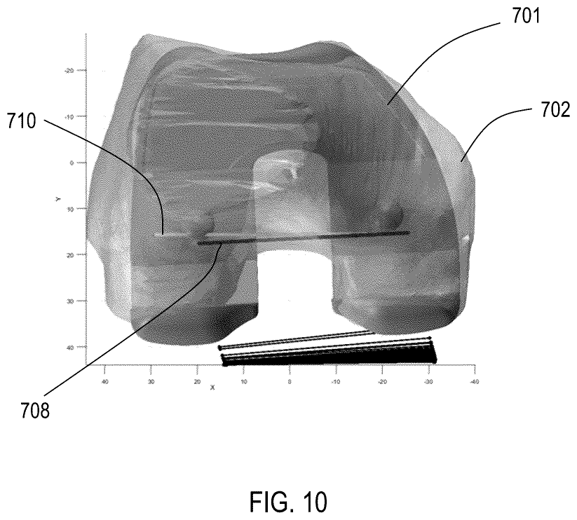

FIGS. 9 and 10 show a graphical representation of step 608 for calculating the COR of the tibia relative to the femur during knee joint motion. A simplified anatomical model of a femur 702 and a tibia 704 highlighting the relative motion of these structures during the knee's flexion and extension is show in FIG. 9. Best-fit circles 706 are superimposed on the image to represent the arcs of motion traced by anatomical landmarks on tibia 704 relative to femur 702. Circles 706 are derived from tracking medial and lateral resection points during guided motion (e.g., pendulum knee drop or range of motion exercises).

A first line 708 passing through the centers of the best-fit circles represents the suggested COR axis as shown in FIG. 10. Axis 708 reflects the rotational center around which tibia 704 moves relative to femur 702 and serves as the reference axis for implant alignment. A second line 710 represents the original implant axis derived from a default surgical plan. Line 710 serves as a comparative baseline, emphasizing the adjustments needed to align the femoral implant with the COR for optimal knee balancing. A point 714 in FIG. 9 indicates the calculated COR, determined by fitting an axis through the center points of the medial and lateral best-fit circles. The COR is necessary for establishing the alignment of the femoral implant's central axis.

While a knee joint is described in the various methods disclosed herein, the present disclosure can be used for any other joint such as hip, shoulder, ankle, etc. Furthermore, although the embodiments disclosed herein has been described with reference to particular features, it is to be understood that these features are merely illustrative of the principles and applications of the present disclosure. It is therefore to be understood that numerous modifications, including changes in the sizes of the various features described herein, may be made to the illustrative embodiments and that other arrangements may be devised without departing from the spirit and scope of the present disclosure. In this regard, the present disclosure encompasses numerous additional features in addition to those specific features set forth in the paragraphs below. Moreover, the foregoing disclosure should be taken by way of illustration rather than by way of limitation as the present disclosure is defined in the examples of the numbered paragraphs, which describe features in accordance with various embodiments of the disclosure, set forth in the claims below.

1. A method to evaluate a knee joint, the method comprising the steps of:
tracking a knee joint during a patellar tendon reflex using at least one sensor to generate sensor data;
generating image data related to the knee joint during the patellar tendon reflex, and
determining a patellar tendon score from the sensor data and the imaging data,
wherein the patellar tendon score represents a knee joint condition.

2. The method of claim 1, wherein the at least one sensor includes an electromyography (EMG) sensor configured to capture real-time muscle activation data during the patellar tendon reflex.

3. The method of claim 1, wherein the at least one sensor may include an inertial measurement unit (IMU) configured to measure any of knee joint angles, velocity, and acceleration during the patellar tendon reflex.

4. The method of claim 1, wherein generating the image data includes capturing real-time kinematic data of the knee joint using computer vision.

5. The method of claim 1, further comprising the step of calculating a knee joint stiffness parameter from the patellar tendon score.

6. The method of claim 1, wherein the sensor data includes measurements from load cells to determine biomechanical forces acting on the knee joint during the patellar tendon reflex.

7. The method of claim 1, further comprising the step of comparing the patellar tendon score with a pre-operative baseline data to evaluate recovery progress.

8. The method of claim 1, further comprising the step of selecting an implant based on the patellar tendon score.

9. The method of claim 8, wherein the implant is any of a femoral or tibial implant for the knee joint.

10. The method of claim 9, further including the step of positioning the implant based on patellar tendon score.

11. The method of claim 1, wherein the image data includes fluoroscopic images of patellar alignment during the patellar tendon reflex.

12. The method of claim 1, further comprising the step of integrating the patellar tendon score with a database of patellar tendon scores for comparative analysis across patient populations.

13. The method of claim 1, wherein the method is performed pre-operatively, intra-operatively, and post-operatively to provide continuous assessment of the knee joint.

14. The method of claim 1, further comprising the step of outputting the patellar tendon score on a user interface.

15. The method of claim 14, wherein the user interface displays any of a reflex speed, peak extension, and range of motion of the knee joint.

16. The method of claim 1, wherein the at least one sensor includes a wearable device configured to provide real-time feedback during the patellar tendon reflex.

17. A method for balancing a knee joint, the method comprising:
obtaining imaging data of a knee joint;
generating a model of the knee joint;
conducting a motion analysis of the knee joint by tracking relative motion between a tibia and a femur to generate a motion arc data;
determining a center of rotation (COR) axis of the tibia relative to the femur based on the motion arc data;
positioning a femoral trial to align a central axis of the femoral trial with the COR axis;
positioning a tibial trial in a first position;
assessing ligament tension of the knee joint, and
selecting a femoral implant based on a natural joint line of the knee joint.

18. The method of claim 17, further comprising a step of adjusting the tibial trial in six degrees of freedom such that the ligament tension is balanced.

19. The method of claim 17, wherein the imaging data includes CT scans segmented to create a three-dimensional model of the knee joint.

20. The method of claim 17, wherein the step of conducting a motion analysis includes tracking points on the tibia relative to the femur during a pendulum knee drop.
